(19) United States
(12) Patent Application Publication
(10) Pub. No.: US 2025/0253026 A1
(43) Pub. Date: Aug. 7, 2025

(54) DEVICES, SYSTEMS, AND METHODS TO CREATE CUSTOMIZED MESSAGES BASED ON CONTEXTUAL INFORMATION

(71) Applicant: iFIT Inc., Logan, UT (US)

(72) Inventor: Chase Brammer, Providence, UT (US)

(21) Appl. No.: 19/012,043

(22) Filed: Jan. 7, 2025

Related U.S. Application Data
(60) Provisional application No. 63/550,323, filed on Feb. 6, 2024.

(51) Int. Cl. G16H 20/30; G06N 20/00

(52) U.S. Cl. CPC G16H 20/30; G06N 20/00

(57) ABSTRACT

A method for creating a customized message to a user is provided. The method includes receiving a request to create a customized message including user data. The method further includes retrieving, historical health data that correlate with the user data. The method further includes generating a contextual prompt for a model wherein the contextual prompt is based on the user data and the historical health data that correlates with the user data, and providing the contextual prompt to the model, wherein the contextual prompt causes the model to create the customized message.

### CROSS-REFERENCE

This application claims the benefit and priority to U.S. Patent Application No. 63/550,323, filed Feb. 6, 2024, which is incorporated herein by reference in its entirety for all that it discloses.

### BACKGROUND

Health is a critical part of a person's well-being. People cultivate their health through health actions, including exercise, diet, lifestyle, and so forth. People may use exercise systems to facilitate their health journey. Conventional exercise systems may provide health recommendations to a person based on pre-determined responses to the person's requests. For example, a user that desires to lose weight and participate in a 5 kilometer race may receive a pre-determined diet and exercise system. But such pre-determined systems are inflexible. Indeed, such plans may be inapplicable to some user's lifestyle, attitude, motivational styles, and so forth.

Mobile applications and/or exercise devices may include various different exercise programs a user may choose to perform while the mobile application and/or the exercise device keeps track of the performance. The exercise programs are often stored in an exercise program library. But exercise program libraries may store massive amounts of exercise programs. This may make identifying, searching, and finding exercise programs of interest to a user difficult. Additionally, some exercise programs may not be as beneficial to a user as other exercise programs, making the selection progress more difficult for the user.

### SUMMARY

In some aspects, the techniques described herein relate to a method for creating a customized message to a user. The method includes receiving a request to generate the customized message to the user. The method further includes receiving user data. The method further includes retrieving historical health data that correlate with the user data. The method further includes generating a contextual prompt for a model, the contextual prompt is based on the user data and the historical health data that correlates with the user data. The method further includes providing the contextual prompt to the model, the contextual prompt causes the model to create the customized message.

In some aspects, the techniques described herein relate to a method for training a model. The method includes receiving a plurality of historical health data, the plurality of historical health data is divided into plurality of subsets. The method further includes providing the plurality of subsets as input for the model, the model including plurality of parameters. The method further including performing a validation for the model, where performing a validation for the model includes one or more of a cross-validation, a consistency check, or a physical model evaluation. The method further including finetuning the model by adjusting one or more of the plurality of parameters and validating the model when all subsets of the plurality of historical health data match with an output received from the model.

This summary is provided to introduce a selection of concepts that are further described below in the detailed description. This summary is not intended to identify key or essential features of the claimed subject matter, nor is it intended to be used as an aid in limiting the scope of the claimed subject matter. Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a schematic representation of system for creating a customized message, according to at least one embodiment of the present disclosure.

FIG. 2 is a representation of a string diagram of creating a customized message to a user, according to at least one embodiment of the present disclosure.

FIG. 3 illustrates a flowchart of a series of acts or a method for creating customized messages to a user, according to at least one embodiment of the present disclosure.

FIG. 4 is a schematic representation of system for training a model to create customized messages, according to at least one embodiment of the present disclosure.

FIG. 5 illustrates a flowchart of a series of acts or a method for creating customized messages to a user, according to at least one embodiment of the present disclosure.

FIG. 6 is a schematic representation of a computing system, according to at least one embodiment of the present disclosure.

### DETAILED DESCRIPTION

This disclosure generally relates to devices, systems, and methods for creating a customized message for a user during an exercise activity. A computing device, such as a retrieval augmented generation (RAG) node, may receive user data from an exercise device and/or mobile device and use the received user data to retrieve relevant historical health data. For example, the RAG may retrieve historical health data that correlates with the received user data. The method further includes generating a contextual prompt based on the user data and the historical health data that correlates with the user data. The contextual prompt may then be provided to a model, such as a generative AI model (e.g., LLM model) to create the customized message. One possible advantage of using a RAG to generate a contextual prompt for a generative AI model is that the model may produce more accurate outputs (e.g., messages).

Typically, a large language model (LLM) includes static training data. This may result in the LLM model's knowledge being limited to the static training data it has received. Based on the date of the training data, many conventional LLM models have a cut-off date on the knowledge it is trained on and/or has access to. This may result in an output from a conventional LLM model that may include false, or outdated information. One possible advantage of utilizing a RAG node to facilitate the creation of customized messages to a user is that the RAG node may provide additional, on spot knowledge, to an LLM model. For example, the RAG may provide information on what field of knowledge the LLM may utilize for the output it generates. The LLM model may consider this additional knowledge when the LLM model provides their output (e.g., the message). For example, the RAG may redirect the LLM model to provide more accurate outputs for each prompt separately the RAG provides to the LLM. For example, a customized message to a person A may be different than a customized message to a person B, due to the different user data related to each user, but also due to the retrieved historical health data that is going to be different for each particular user based what historical health data is correlating with each user's user data.

In addition, re-training an LLM model with all the knowledge base the RAG has access to takes time and money as the LLM model may need to be finetuned a plurality of times, and continuously, to be up-to-date all the time. One possible advantage of the system using a RAG is that the RAG may gather the additional relevant data and provide only that additional relevant data to the LLM together with a more sophisticated prompt on executing a creation of a customized message. In this manner, utilizing a RAG may be cheaper and more efficient than training an LLM model and/or re-training an LLM model utilizing the additional relevant data.

As illustrated by the foregoing discussion, the present disclosure utilizes a variety of terms to describe features and advantages of the context generation system. Additional detail is now provided regarding the meaning of a number of these terms.

For example, as used herein, a “model” refers to a generative AI model or a large language model (LLM) that is trained to generate an output in response to an input based on a large dataset. A model may include a neural network having a significant number of parameters (e.g., billions of parameters) that the model can consider in performing a task or otherwise generating an output based on an input. In one or more embodiments described herein, a model is trained to generate a response to a prompt. The model may be trained with health science, behavior science, and general exercise information. For example, the model may be trained to provide customized training suggestions for a particular user based on their training and health history. In one or more implementations described herein, the model refers specifically to an LLM, though other types of models may be used in generating responses to prompts. Indeed, while one or more embodiments described herein refer to features associated with generating contextual prompts for a LLM, similar features may apply to determining and/or generating contextual prompts for other types of models. One difference between a machine learning (ML) model and a generative AI model is that the ML model may only select an output from the plurality of output options provided to it, whereas a generative AI model is able to generate a new output and modify or adapt a provided output option.

For example, as used herein, a “retrieval augmented generation” or “RAG” refers to an AI framework that allows a generative AI model to access external information not included in its training data. In one or more embodiments, utilizing a RAG, a LLM model may access up-to-date information, proprietary information, or personal information not readily available elsewhere, to provide better responses to prompts. For example, a LLM model may request and/or receive user specific information (such as prior training history or injuries) to generate customized responses for that particular user's needs. In some embodiments, a RAG may decide what additional contextual information to provide together with a prompt. For example, a RAG may decide to select data that is relevant to, related to, or correlates with another set of data. In some embodiments, a RAG may select to provide a subset of historical health data to a model wherein the subset of historical health data correlates with, relates to, or is relevant to a received user data.

As used herein, “contextual information” may be information that may be used by a model that directs the model to generate a relevant or more accurate response to a prompt. A contextual information may include information related to a prompt that is not directly stated in the prompt. As used herein, “contextual prompt” may include contextual information and a prompt (e.g., a query, a task, or a request) that a model may use to create a more accurate output (e.g., a message). In one or more embodiments described herein, a contextual prompt is generated based on an individual user's data and historical health data that correlates with the individual user's data. For example, the individual user's data may be the user's exercise data and the historical health data may be exercising programs that correlates with the individual user's data. Contextual information may be identified through a variety of mechanisms. For example, contextual information may be identified using a variety of correlation techniques to identify a correlation metric. Determining a correlation metric between user data and the historical health data may involve, for example, comparing user's exercise data to a catalog of various different exercises and identifying similarities between them.

As used herein, an “exercise devices” may refer to any type of exercise device, such as a treadmill, an elliptical device, a stationary bicycle, a rower, a cable extension device, a mirror including a backlit display behind a mirrored surface, any other exercise device, and combinations thereof.

As used herein, an “exercise program” may include an exercise video, an animation, written instructions, verbal instructions, images, or a combination thereof. In one or more embodiments, the exercise video or the animation may include a representation of a trainer that is providing instruction for the user to perform a workout. In some examples, the trainer in the exercise video or in the animation may be performing the workout in the exercise video or in the animation. The exercise program may be displayed on a display of an exercise device, displayed on a display of a mobile device, provided through a speaker, or a combination thereof.

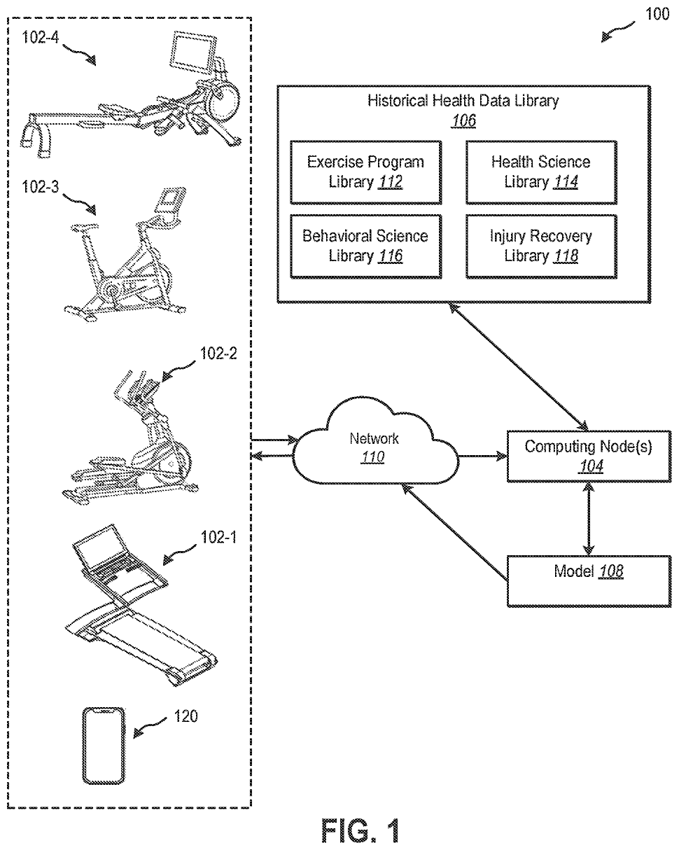

FIG. 1 is a schematic representation of a system 100 for creating a customized message, according to at least one embodiment of the present disclosure. The system 100 may include one or more exercise devices (collectively 102). The exercise devices 102 may include any type of exercise device, such as a treadmill 102-1, an elliptical device 102-2, a stationary bicycle 102-3, a rower 102-4, a cable extension device, a mirror including a backlit display behind a mirrored surface, any other exercise device, and combinations thereof. While specific exercise devices are illustrated and discussed herein, it should be understood that the techniques of the present disclosure may be applied to any exercise device that is capable of implementing an exercise program. The system 100 may further include a mobile device 120, such as a mobile phone, a laptop, a tablet, or a smart watch. The exercise devices 102 and/or the mobile device 120 may be in communication with computing node(s) 104 over a network 110. The network 110 may be any type of network. For example, the network 110 may be a local area network (LAN), a wide area network (WAN), a Wi-Fi network, a cellular network, any other network, and combinations thereof.

The system 100 may include a historical health data library 106. The historical health data library 106 may further include an exercise program library 112. The exercise program library 112 may include a repository of one or more exercise programs. The exercise programs may be implemented on one or more of the exercise devices 102 or the mobile device 120. In some embodiments, the exercise programs are specifically tailored to a particular exercise device 102. For example, the exercise programs may be specifically tailored to a type of exercise device 102, such as the elliptical device 102-2. In some embodiments, the exercise programs are usable by more than one type of exercise device 102. For example, an exercise program may include resistance settings for a flywheel, and the exercise program may be usable by any flywheel-based exercise device 102. In some embodiments, the exercise programs are not tailored to be performed with an exercise machine. For example, the exercise program may be displayed on a mobile device 120 and performed by the user without any exercise device 102.

The historical health data library 106 may further include a behavioral science library 116. The behavioral science library 116 may include behavioral science information relating to motivation, habits, social influence, other types of behavioral science information, or a combination thereof. For example, the behavioral science library 116 may include research papers, research studies, books, and any other sources of information on behavioral science. The historical health data library 106 may further include health science library 114. The health science library 114 may include information relating to nutrition and exercise science (e.g., mechanics of movement, anatomy, exercise physiology, exercise psychology, and biomechanics). For example, the health science library 114 may include research papers, research studies, books, and any other sources of information on nutrition and/or exercise science.

The historical health data library 106 may further include injury recovery library 118. The injury recovery library 118 may include anatomy of injury information, rehabilitation information, kinesiology, injury treatment plans, physical therapy information, or a sports medicine information. For example, the injury recovery library 118 may include research papers, research studies, books, and any other sources of information on injury recovery.

The system 100 may include one or more computing nodes 104. The one or more computing nodes 104 may be a retrieval augmented generation (RAG) node. The RAG node may be used to improve an output of a large language model (LLM) by providing a knowledge base to an internal database, such as the historical health data library 106. The computing node 104 may also collect information from the one or more exercise devices 102 and the mobile device 120. In some embodiments, the computing node 104 may receive user data from the one or more exercise devices 102 and/or the mobile device 120. The user data may include one or more of a training history, a goal, patterns in training, an activity level, injuries, strengths, weaknesses, motivation information, or a combination thereof. In some embodiments, patterns in training may include one or more of a time spent on an activity, a time between activities, preferred weekdays for performing an activity, preferred hours for performing an activity, or a combination thereof. In some embodiments, the training history may include all activities and/or exercises performed by the user. In some embodiments, the training history may include all exercises performed by the user in the last three months. In some embodiments, the training history may include all exercises performed after a goal was set by the user. In some embodiments, the strengths and/or weaknesses are identified by the user. In some embodiments, the strengths and weaknesses are automatically identified by an exercise device and/or a mobile device.

For example, user data may include that the user likes to run 5K every Saturday morning, that it takes them 30 min on average, that they're recovering from a toe surgery, and that their goal is to run 10K under 60 min. In another example, the user data may include that the user's strength is short sprints while their weakness is long extended runs. In yet another example, the user data may include motivation information, such as, what type of motivational messages work best on improving the user's motivation.

In some embodiments, the one or more exercise devices 102 and/or the mobile device 120 may send a request to the computing node 104 to generate a customized message to the user. For example, the customized message may be a customized activity suggestion for the user. A customized activity suggestion may include one or more different exercises, on one or more exercise devices, one or more intensity level for the exercise, to be performed on a specific date and time (e.g., start time), and/or for a specific length of time. In some embodiments, the customized activity suggestion may further include dietary information and recovery information. For example, the customized activity suggestion may include meal plans before, during, and after the exercise and a recommended amount of recovery time after the exercise.

In some embodiments, the customized message may be a customized motivational message to the user. For example, a customized motivational message may include personalized message to the user for motivating the user to exercise. In some examples, the motivational message may include behavioral advice. In some examples, the motivational message may include encouraging information. In some examples, the motivational message may include exercise recommendations. In some examples, the motivational message may include information related to other users. For example, the motivational message may include information related to other users' experiences in the same or a similar situation.

In some embodiments, the computing node 104 is configured to generate a contextual prompt that is based on the received user data from one or more exercise devices 102 or the mobile device 120, and the historical health data received from the historical health science library 106. The contextual prompt may include information related to the prompt that is not directly stated in the prompt. In one or more embodiments described herein, the contextual prompt is generated based on an individual user's data and historical health data that correlates with the individual user's data. The contextual prompt may be generated through a variety of mechanisms. For example, the contextual prompt may be generated by identifying correlations by using a variety of correlation techniques to identify a correlation metric. Determining a correlation metric between user data and historical health data may involve, for example, comparing the user's exercise data to a catalog of various different exercises and identifying similarities between them.

The system 100 may further include a model 108. In some embodiments, the model 108 is a generative AI model configured to generate an output based on an input. In some embodiments, the model 108 is a LLM model that is trained on a large dataset. The model 108 may include a neural network having a significant number of parameters (e.g., billions of parameters) that the model 108 can consider in performing a task or otherwise generating an output based on an input. In some embodiments, the model 108 is trained to generate a response to a prompt. The model 108 may be trained with health science, behavior science, injury recover information, exercise information, or a combination thereof. For example, the model may be trained to provide customized activity suggestions for a particular user based on their user data and historical health data. In one or more implementations described herein, the model 108 refers specifically to an LLM, though other types of models may be used in generating responses to prompts. While one or more embodiments described herein refer to features associated with generating contextual prompts for a LLM, similar features may apply to determining and/or generating contextual prompts for other types of models.

In some embodiments, the model 108 is configured to receive the contextual prompt from the computing node 104. The model 108 may process the prompt, and the contextual information included in the prompt to create a customized message. The model 108 may then deliver the customized message to one or more exercise devices 102 and/or the mobile device 120. In some embodiments, the model 108 may deliver the customized message to the computing node 104, and the computing node 104 delivers it to the user. In some embodiments, the model 108 is configured to generate a new activity, by modifying, adapting, or combining one or more activities stored on the exercise program library 112.

The one or more exercise devices 102 and/or the mobile device 120 may then display the customized message to the user via a display. In some embodiments, receiving the customized message may cause the exercise device 120 to display the customized message on a display of the exercise device 102. In some embodiments, receiving the customized message may cause the exercise device 102 to display the customized message on a display of the exercise device and to adjust the operating parameters of the exercise device 102 based on the customized message. One possible benefit of delivering a customized message to a user is that the user is provided with an activity suggestion that takes into account user's injuries, goals, strengths, weaknesses, preferences, etc. and provides a customized activity backed up with health data science, behavioral science, and injury recovery information.

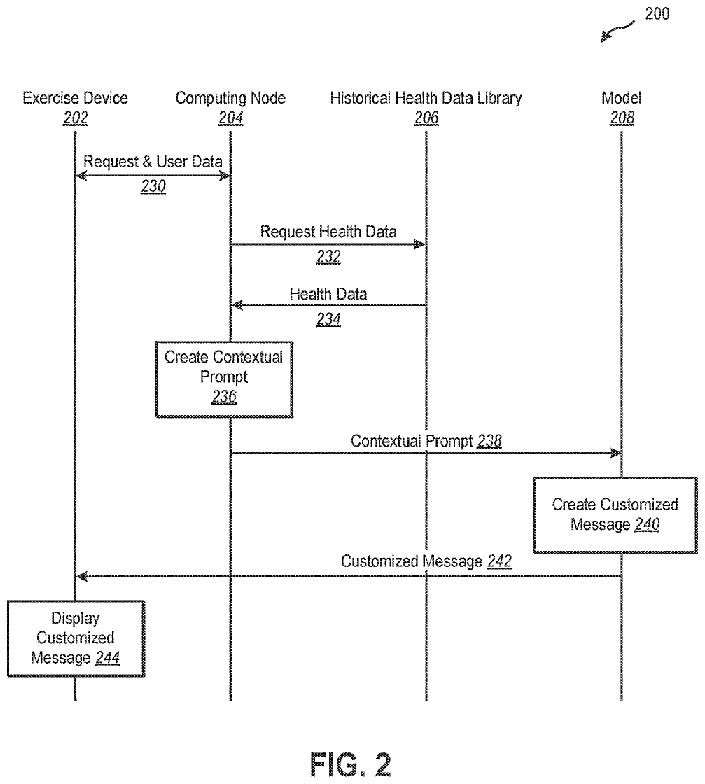

FIG. 2 is a representation of a string diagram 200 of creating a customized message to a user, according to at least one embodiment of the present disclosure. An exercise device 202 may transmit a request 230 to generate a customized message to a user. In some embodiments, the request 230 further includes user data. For example, the user data may include one or more of a training history, a goal, patterns in training, an activity level, injuries, strengths, weaknesses, motivation information, or a combination thereof.

A computing node 204 may receive the request 230 including the user data. The computing node 204 may be a retrieval augmented generation (RAG) node. The computing node 204 may, upon receiving the request 230 retrieve historical health data from a historical health data library 206. For example, the computing node 204 may send a request 232 for historical health data to the historical health data library 206. The historical health data may include one or more of exercise program information, behavioral science information, health science information, and injury recovery information. The historical health data library 206 may send historical health data 234 to the computing node 204 in response to receiving the request for health data 234. In some embodiments, the computing node 204 requests only health data that correlates with the user data received with the request 230. In some embodiments, the computing node 204 requests all historical health data and the computing node 204 determines the correlation between the user data and the historical health data after receiving the health data 234.

The computing node 204 may then generate a contextual prompt 236 that is based on the received user data from the exercise devices 202, and the received historical health data 234 received from the historical health data library 206. In one or more embodiments described herein, a contextual prompt is generated 236 based on an individual user's data and historical health data that correlates with the individual user's data. The contextual prompt may be generated through a variety of mechanisms. For example, the contextual prompt may be generated by identifying correlations by using a variety of correlation techniques to identify a correlation metric. Determining a correlation metric between the user data and historical health data may involve, for example, comparing the user's exercise data to a catalog of various different exercises and identifying similarities between them. In another example, it may involve comparing the user's prior successful motivation messages to behavioral science information related to motivation. In a further example, it may involve comparing the user's injury information to injury recovery information. In yet another example, it may involve comparing the user's goal to a nutritional information or other health science information.

The computing node 204 may then provide the contextual prompt 238 to a model 208. The model 208 may be a generative AI model configured to generate a customized message 240 based on an input. In some embodiments, the model 208 is a LLM model that is trained on a large dataset. The model 208 may include a neural network having a significant number of parameters (e.g., billions of parameters) that the model 208 can consider in performing a task or otherwise generating an output based on the contextual prompt 238. The customized message 240 created by the model 208 may include a customized activity suggestion, a customized motivational message, or a combination thereof.

The model 208 may then deliver the customized message 242 to the exercise device 202. In some embodiments, the model 208 delivers the customized message 242 directly to the exercise device 202. In some embodiments, the model 208 delivers the customized message 242 to the computing node 204, and the computing node 204 delivers the customized message 242 to the exercise device 202. In some embodiments, receiving the customized message 242 may cause the exercise device 202 to display the customized message 244 on a display of the exercise device 202. In some embodiments, receiving the customized message 242 may cause the exercise device 202 to display the customized message 244 on a display of the exercise device 202 and to adjust the operating parameters of the exercise device 202 based on the customized message.

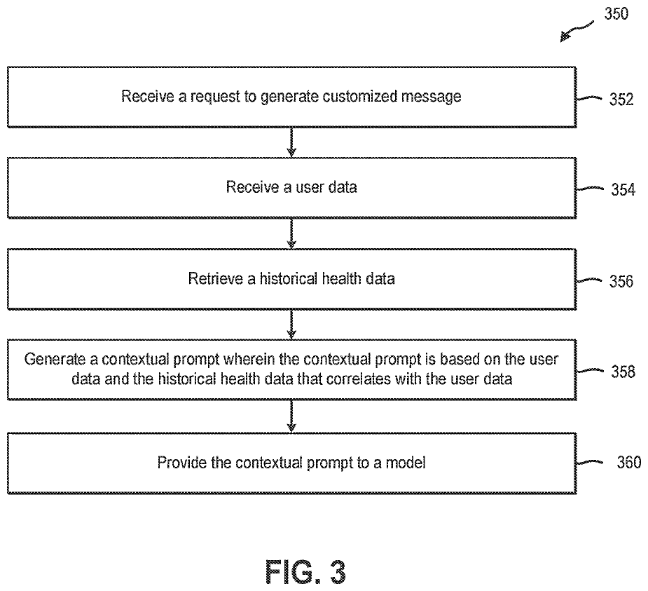

FIG. 3 illustrates a flowchart of a series of acts 350 or a method for creating customized messages to a user, according to at least one embodiment of the present disclosure. While FIG. 3 illustrates acts according to one embodiment, alternative embodiments may omit, add to, reorder, and/or modify any of the acts shown in FIG. 3. The acts of FIG. 3 can be performed as part of a method. Alternatively, a computer-readable medium can comprise instructions that, when executed by one or more processors, cause a computing device to perform the acts of FIG. 3. In some embodiments, a system can perform the acts of FIG. 3.

As shown in FIG. 3, the series of acts 350 may include an act 352 of receiving a request to generate a customized message. In some embodiments, the request may be received from a mobile device or an exercise device. In some embodiments, the request is received by a computing node. For example, the computing node may be a retrieval augmented generation (RAG) node.

The series of acts 350 may further include an act 354 of receiving user data. In some embodiments, the request to generate a customized message is received together with the user data. In some embodiments, the user data may include one or more of a training history, a goal, patterns in training, an activity level, injuries, strengths, weaknesses, motivation information, or a combination thereof.

The series of acts 350 may further include an act 356 of retrieving historical health data. In some embodiments, the historical health data may include one or more of exercise program information, behavioral science information, health science information, and injury recovery information.

The series of acts 350 may further include an act 358 of generating a contextual prompt wherein the contextual prompt is based on the user data and the historical health data that correlates with the user data. For example, contextual information for the contextual prompt may be identified using a variety of correlation techniques to identify a correlation metric. Determining a correlation metric between user data and historical health data may involve, for example, comparing the user's exercise data to a catalog of various different exercises and identifying similarities between them.

The series of acts 350 may further include an act 360 of providing the contextual prompt to a model. In some embodiments, the model is a generative AI model configured to generate an output based on the contextual prompt. In some embodiments, the model is a LLM model that is trained on a large dataset. In some embodiments, the model may include a neural network having a significant number of parameters (e.g., billions of parameters) that the model can consider in performing a task or otherwise generating an output based on the prompt.

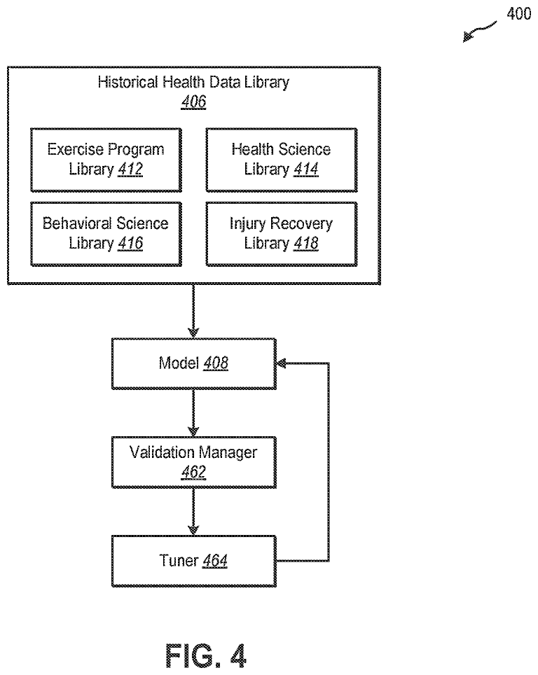

FIG. 4 is a schematic representation of a system 400 for training a model to create customized messages, according to at least one embodiment of the present disclosure. The system 400 may include a historical health data library 406. The historical health data library 406 may further include an exercise program library 412. The exercise program library 412 may include a repository of one or more exercise programs. The exercise programs may be implemented on one or more exercise devices or on a mobile device. In some embodiments, the exercise programs are specifically tailored to a particular exercise device. For example, the exercise programs may be specifically tailored to a type of exercise device, such as an elliptical. In some embodiments, the exercise programs are usable by more than one type of exercise device. For example, an exercise program may include resistance settings for a flywheel, and the exercise program may be usable by any flywheel-based exercise device. In some embodiments, the exercise programs are not tailored to be performed with an exercise machine. For example, the exercise program may be displayed on a mobile device and performed by the user without any exercise machine.

The historical health data library 406 may further include a behavioral science library 416. The behavioral science library 416 may include behavioral science information relating to motivation, habits, social influence, other types of behavioral science information, or a combination thereof. For example, the behavioral science library 416 may include research papers, research studies, books, and any other sources of information on behavioral science. The historical health data library 406 may further include a health science library 414. The health science library 414 may include information relating to nutrition and exercise science (e.g., mechanics of movement, anatomy, exercise physiology, exercise psychology, and biomechanics). For example, the health science library 414 may include research papers, research studies, books, and any other sources of information on nutrition and/or exercise science.

The historical health data library 416 may further include an injury recovery library 418. The injury recovery library 418 may include anatomy of injury information, rehabilitation information, kinesiology, injury treatment plans, physical therapy information, or a sports medicine information. For example, the injury recovery library 418 may include research papers, research studies, books, and any other sources of information on injury recovery. The system 400 may use the data stored on the historical health data library 406 to train the model 408 with exercise, health science, behavioral science, and injury recovery information. In one or more embodiments, the data stored on the historical health data library 406 may be preprocessed before being delivered to the model 408. For example, the data may be cleaned, tokenized, split into training sets or validation sets, or a combination thereof.

The system 400 further includes a model 408. In some embodiments, the model 408 is a generative AI model. In some embodiments, the model 408 is a LLM model. The model 408 may include a neural network having a significant number of parameters (e.g., billions of parameters) that the model 408 can consider in performing a task or otherwise generating an output based on an input. In some embodiments, the model 408 is trained to generate a response to a prompt. As shown in FIG. 4, the model 408 is trained with health science, behavior science, injury recover information, exercise information, or a combination thereof. In some embodiments, the model 408 creates new parameters consistent with the new data provided by the historical health data library 406.

The system 400 further includes a validation manager 462. The validation manager 462 may validate the model 408 by utilizing cross-validation, consistency checks, and physical model evaluations to help to improve the validation and verification of the model. For example, based on the results of at least one of the cross-validation, consistency checks, and physical model evaluations, the model may be finetuned.

The system 400 further includes a tuner 464. The tuner 464 may tune one or more parameters of the model 408. The parameters of the model 408 may be the parameters in the model 408 that control the learning process. Other parameters of the model 408 may be adjusted or derived during training of the model 408. For example, the tuner 464 may identify which portions of the output received during validation of a sub set of the historical health data does not correspond to the input of historical health data subset. The tuner 464 may determine which parameter(s) may adjust the output. Adjusting or tuning the parameters may adjust the output. The tuner 464 may adjust the parameters of the model 408 until the validation manager 462 successfully validates all subsets of the historical health data.

In some embodiments, the trained model 408 is configured to generate a new activity, by modifying, adapting, or combining one or more activities used for training the model 408. In some embodiments, a trained model 408 may be used without a RAG. For example, a trained model 408 that includes the knowledge of a user data and historical health data may not require a RAG to provide any additional contextual information or a prompt for the trained model 408 to create a customized message. In some embodiments, a trained model 408 may be used together with a RAG. For example, the trained model 408 may be trained with the historical health data, while the RAG may still provide contextual prompt to the trained model 408 that includes the user data.

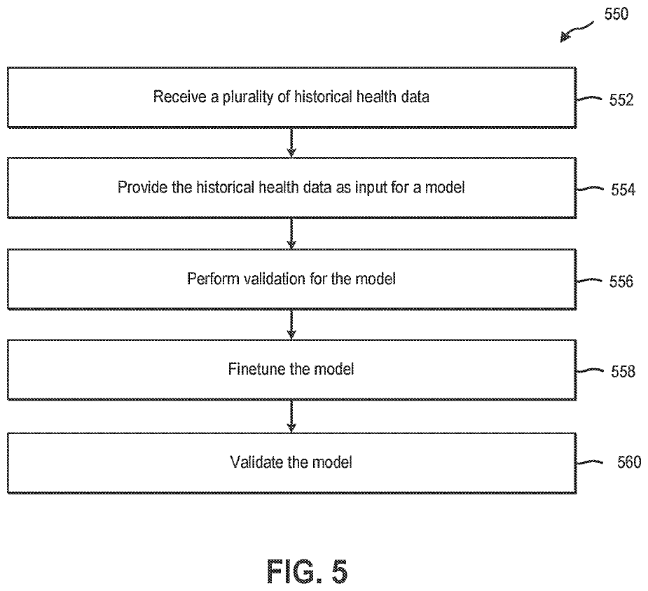

FIG. 5 illustrates a flowchart of a series of acts 550 or a method for creating customized messages to a user, according to at least one embodiment of the present disclosure. While FIG. 5 illustrates acts according to one embodiment, alternative embodiments may omit, add to, reorder, and/or modify any of the acts shown in FIG. 5. The acts of FIG. 5 can be performed as part of a method. Alternatively, a computer-readable medium can comprise instructions that, when executed by one or more processors, cause a computing device to perform the acts of FIG. 5. In some embodiments, a system can perform the acts of FIG. 5.

As shown in FIG. 5, the series of acts 550 may include an act 552 of receiving a plurality of historical health data. In some embodiments, the plurality of historical health data is divided into a plurality of subsets.

The series of acts 550 may further include an act 554 of providing the historical health data as input for a model. In some embodiments, the historical health data may be preprocessed before provided as input for the model. The model may include plurality of parameters.

The series of acts 550 may further include an act 556 of performing validation for the model. In some embodiments, performing validation for the model includes one or more of a cross-validation, a consistency check, or a physical model evaluation.

The series of acts 550 may further include an act 558 of finetuning the model. For example, finetuning the model may include adjusting one or more of the plurality of parameters.

The series of acts 550 may further include an act 560 of validating the model. In some embodiments, the model is validated when all subsets of the plurality of historical health data match with an output received from the model.

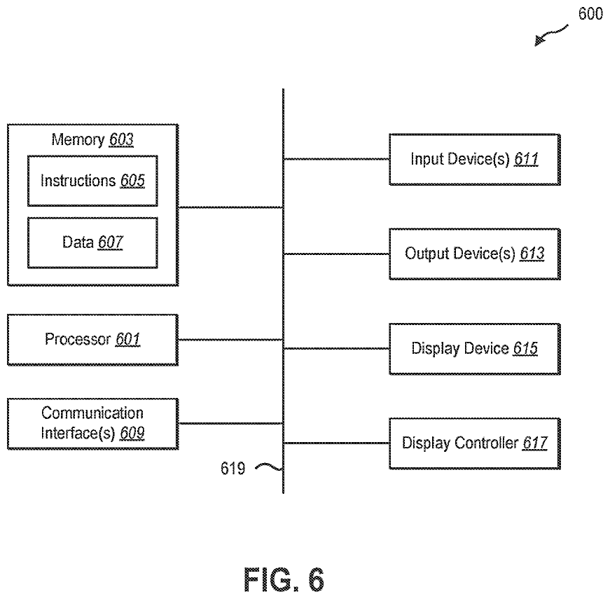

FIG. 6 illustrates certain components that may be included within a computer system 600, such as the computing node 104 of FIG. 1 or computing node 204 of FIG. 2. One or more computer systems 600 may be used to implement the various devices, components, and systems described herein.

The computer system 600 includes a processor 601. The processor 601 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 601 may be referred to as a central processing unit (CPU). Although just a single processor 601 is shown in the computer system 600 of FIG. 6, in an alternative configuration, a combination of processors (e.g., an ARM and DSP) could be used.

The computer system 600 also includes memory 603 in electronic communication with the processor 601. The memory 603 may be any electronic component capable of storing electronic information. For example, the memory 603 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 605 and data 607 may be stored in the memory 603. The instructions 605 may be executable by the processor 601 to implement some or all of the functionality disclosed herein. Executing the instructions 605 may involve the use of the data 607 that is stored in the memory 603. Any of the various examples of modules and components described herein may be implemented, partially or wholly, as instructions 605 stored in memory 603 and executed by the processor 601. Any of the various examples of data described herein may be among the data 607 that is stored in memory 603 and used during execution of the instructions 605 by the processor 601.

A computer system 600 may also include one or more communication interfaces 609 for communicating with other electronic devices. The communication interface(s) 609 may be based on wired communication technology, wireless communication technology, or both. Some examples of communication interfaces 609 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 600 may also include one or more input devices 611 and one or more output devices 613. Some examples of input devices 611 include a keyboard, mouse, microphone, remote control device, button, joystick, trackball, touchpad, and lightpen. Some examples of output devices 613 include a speaker and a printer. One specific type of output device that is typically included in a computer system 600 is a display device 615. Display devices 615 used with embodiments disclosed herein may utilize any suitable image projection technology, such as liquid crystal display (LCD), light-emitting diode (LED), gas plasma, electroluminescence, or the like. A display controller 617 may also be provided, for converting data 607 stored in the memory 603 into text, graphics, and/or moving images (as appropriate) shown on the display device 615.

The various components of the computer system 600 may be coupled together by one or more buses, which may include a power bus, a control signal bus, a status signal bus, a data bus, etc. For the sake of clarity, the various buses are illustrated in FIG. 6 as a bus system 619.

### INDUSTRIAL APPLICABILITY

This disclosure generally relates to devices, systems, and methods for creating a customized message for a user during an exercise activity. A computing device, such as a retrieval augmented generation (RAG) node, may receive user data from an exercise device and/or mobile device and use the received user data to retrieve relevant historical health data. For example, the RAG may retrieve historical health data that correlates with the received user data. The method further includes generating a contextual prompt based on the user data and the historical health data that correlates with the user data. The contextual prompt may then be provided to a model, such as a generative AI model (e.g., LLM model) to create the customized message. One possible advantage of using a RAG to generate a contextual prompt for a generative AI model is that the model may produce more accurate outputs (e.g., messages).

Typically, a large language model (LLM) includes static training data. This may result in the LLM model's knowledge being limited to the static training data it has received. Based on the date of the training data, many conventional LLM models have a cut-off date on the knowledge it is trained on and/or has access to. This may result in an output from a conventional LLM model that may include false, or outdated information. One possible advantage of utilizing a RAG node to facilitate the creation of customized messages to a user is that the RAG node may provide additional, on spot knowledge, to an LLM model. For example, the RAG may provide information on what field of knowledge the LLM may utilize for the output it generates. The LLM model may consider this additional knowledge when the LLM model provides their output (e.g., the message). For example, the RAG may redirect the LLM model to provide more accurate outputs for each prompt separately the RAG provides to the LLM. For example, a customized message to a person A may be different than a customized message to a person B, due to the different user data related to each user, but also due to the retrieved historical health data that is going to be different for each particular user based what historical health data is correlating with each user's user data.

In addition, re-training an LLM model with all the knowledge base the RAG has access to takes time and money as the LLM model may need to be finetuned a plurality of times, and continuously, to be up-to-date all the time. One possible advantage of the system using a RAG is that the RAG may gather the additional relevant data and provide only that additional relevant data to the LLM together with a more sophisticated prompt on executing a creation of a customized message. In this manner, utilizing a RAG may be cheaper and more efficient than training an LLM model and/or re-training an LLM model utilizing the additional relevant data.

As illustrated by the foregoing discussion, the present disclosure utilizes a variety of terms to describe features and advantages of the context generation system. Additional detail is now provided regarding the meaning of a number of these terms.

For example, as used herein, a “model” refers to a generative AI model or a large language model (LLM) that is trained to generate an output in response to an input based on a large dataset. A model may include a neural network having a significant number of parameters (e.g., billions of parameters) that the model can consider in performing a task or otherwise generating an output based on an input. In one or more embodiments described herein, a model is trained to generate a response to a prompt. The model may be trained with health science, behavior science, and general exercise information. For example, the model may be trained to provide customized training suggestions for a particular user based on their training and health history. In one or more implementations described herein, the model refers specifically to an LLM, though other types of models may be used in generating responses to prompts. Indeed, while one or more embodiments described herein refer to features associated with generating contextual prompts for a LLM, similar features may apply to determining and/or generating contextual prompts for other types of models. One difference between a machine learning (ML) model and a generative AI model is that the ML model may only select an output from the plurality of output options provided to it, whereas a generative AI model is able to generate a new output and modify or adapt a provided output option.

For example, as used herein, a “retrieval augmented generation” or “RAG” refers to an AI framework that allows a generative AI model to access external information not included in its training data. In one or more embodiments, utilizing a RAG, a LLM model may access up-to-date information, proprietary information, or personal information not readily available elsewhere, to provide better responses to prompts. For example, a LLM model may request and/or receive user specific information (such as prior training history or injuries) to generate customized responses for that particular user's needs. In some embodiments, a RAG may decide what additional contextual information to provide together with a prompt. For example, a RAG may decide to select data that is relevant to, related to, or correlates with another set of data. In some embodiments, a RAG may select to provide a subset of historical health data to a model wherein the subset of historical health data correlates with, relates to, or is relevant to a received user data.

As used herein, “contextual information” may be information that may be used by a model that directs the model to generate a relevant or more accurate response to a prompt. A contextual information may include information related to a prompt that is not directly stated in the prompt. As used herein, “contextual prompt” may include contextual information and a prompt (e.g., a query, a task, or a request) that a model may use to create a more accurate output (e.g., a message). In one or more embodiments described herein, a contextual prompt is generated based on an individual user's data and historical health data that correlates with the individual user's data. For example, the individual user's data may be the user's exercise data and the historical health data may be exercising programs that correlates with the individual user's data. Contextual information may be identified through a variety of mechanisms. For example, contextual information may be identified using a variety of correlation techniques to identify a correlation metric. Determining a correlation metric between user data and the historical health data may involve, for example, comparing user's exercise data to a catalog of various different exercises and identifying similarities between them.

As used herein, an “exercise devices” may refer to any type of exercise device, such as a treadmill, an elliptical device, a stationary bicycle, a rower, a cable extension device, a mirror including a backlit display behind a mirrored surface, any other exercise device, and combinations thereof.

As used herein, an “exercise program” may include an exercise video, an animation, written instructions, verbal instructions, images, or a combination thereof. In one or more embodiments, the exercise video or the animation may include a representation of a trainer that is providing instruction for the user to perform a workout. In some examples, the trainer in the exercise video or in the animation may be performing the workout in the exercise video or in the animation. The exercise program may be displayed on a display of an exercise device, displayed on a display of a mobile device, provided through a speaker, or a combination thereof.

A schematic representation of a system for creating a customized message is provided. The system may include one or more exercise devices. The exercise devices may include any type of exercise device, such as a treadmill, an elliptical device, a stationary bicycle, a rower, a cable extension device, a mirror including a backlit display behind a mirrored surface, any other exercise device, and combinations thereof. While specific exercise devices are illustrated and discussed herein, it should be understood that the techniques of the present disclosure may be applied to any exercise device that is capable of implementing an exercise program. The system may further include a mobile device, such as a mobile phone, a laptop, a tablet, or a smart watch. The exercise devices and/or the mobile device may be in communication with computing node(s) over a network. The network may be any type of network. For example, the network may be a local area network (LAN), a wide area network (WAN), a Wi-Fi network, a cellular network, any other network, and combinations thereof.

The system may include a historical health data library. The historical health data library may further include an exercise program library. The exercise program library may include a repository of one or more exercise programs. The exercise programs may be implemented on one or more of the exercise devices or the mobile device. In some embodiments, the exercise programs are specifically tailored to a particular exercise device. For example, the exercise programs may be specifically tailored to a type of exercise device, such as the elliptical device. In some embodiments, the exercise programs are usable by more than one type of exercise device. For example, an exercise program may include resistance settings for a flywheel, and the exercise program may be usable by any flywheel-based exercise device. In some embodiments, the exercise programs are not tailored to be performed with an exercise machine. For example, the exercise program may be displayed on a mobile device and performed by the user without any exercise machine.

The historical health data library may further include a behavioral science library. The behavioral science library may include behavioral science information relating to motivation, habits, social influence, other types of behavioral science information, or a combination thereof. For example, the behavioral science library may include research papers, research studies, books, and any other sources of information on behavioral science. The historical health data library may further include health science library. The health science library may include information relating to nutrition and exercise science (e.g., mechanics of movement, anatomy, exercise physiology, exercise psychology, and biomechanics). For example, the health science library may include research papers, research studies, books, and any other sources of information on nutrition and/or exercise science.

The historical health data library may further include injury recovery library. The injury recovery library may include anatomy of injury information, rehabilitation information, kinesiology, injury treatment plans, physical therapy information, or a sports medicine information. For example, the injury recovery library may include research papers, research studies, books, and any other sources of information on injury recovery.

The system may include one or more computing nodes. The one or more computing nodes may be a retrieval augmented generation (RAG) node. The RAG node may be used to improve an output of a large language model (LLM) by providing a knowledge base to an internal database, such as the historical health data library. The computing node may also collect information from the one or more exercise devices and the mobile device. In some embodiments, the computing node may receive user data from the one or more exercise devices and/or the mobile device. The user data may include one or more of a training history, a goal, patterns in training, an activity level, injuries, strengths, weaknesses, motivation information, or a combination thereof. In some embodiments, patterns in training may include one or more of a time spent on an activity, a time between activities, preferred weekdays for performing an activity, preferred hours for performing an activity, or a combination thereof. In some embodiments, the training history may include all activities and/or exercises performed by the user. In some embodiments, the training history may include all exercises performed by the user in the last three months. In some embodiments, the training history may include all exercises performed after a goal was set by the user. In some embodiments, the strengths and/or weaknesses are identified by the user. In some embodiments, the strengths and weaknesses are automatically identified by an exercise device and/or a mobile device.

For example, user data may include that the user likes to run 5K every Saturday morning, that it takes them 30 min on average, that they're recovering from a toe surgery, and that their goal is to run 10K under 60 min. In another example, the user data may include that the user's strength is short sprints while their weakness is long extended runs. In yet another example, the user data may include motivation information, such as, what type of motivational messages work best on improving the user's motivation.

In some embodiments, the one or more exercise devices and/or the mobile device may send a request to the computing node to generate a customized message to the user. For example, the customized message may be a customized activity suggestion for the user. A customized activity suggestion may include one or more different exercises, on one or more exercise devices, one or more intensity level for the exercise, to be performed on a specific date and time (e.g., start time), and/or for a specific length of time. In some embodiments, the customized activity suggestion may further include dietary information and recovery information. For example, the customized activity suggestion may include meal plans before, during, and after the exercise and a recommended amount of recovery time after the exercise.

In some embodiments, the customized message may be a customized motivational message to the user. For example, a customized motivational message may include personalized message to the user for motivating the user to exercise. In some examples, the motivational message may include behavioral advice. In some examples, the motivational message may include encouraging information. In some examples, the motivational message may include exercise recommendations. In some examples, the motivational message may include information related to other users. For example, the motivational message may include information related to other users' experiences in the same or a similar situation.

In some embodiments, the computing node is configured to generate a contextual prompt that is based on the received user data from one or more exercise devices or the mobile device, and the historical health data received from the historical health science library. The contextual prompt may include information related to the prompt that is not directly stated in the prompt. In one or more embodiments described herein, the contextual prompt is generated based on an individual user's data and historical health data that correlates with the individual user's data. The contextual prompt may be generated through a variety of mechanisms. For example, the contextual prompt may be generated by identifying correlations by using a variety of correlation techniques to identify a correlation metric. Determining a correlation metric between user data and historical health data may involve, for example, comparing the user's exercise data to a catalog of various different exercises and identifying similarities between them.

The system may further include a model. In some embodiments, the model is a generative AI model configured to generate an output based on an input. In some embodiments, the model is a LLM model that is trained on a large dataset. The model may include a neural network having a significant number of parameters (e.g., billions of parameters) that the model can consider in performing a task or otherwise generating an output based on an input. In some embodiments, the model is trained to generate a response to a prompt. The model may be trained with health science, behavior science, injury recover information, exercise information, or a combination thereof. For example, the model may be trained to provide customized activity suggestions for a particular user based on their user data and historical health data. In one or more implementations described herein, the model refers specifically to a LLM, though other types of models may be used in generating responses to prompts. While one or more embodiments described herein refer to features associated with generating contextual prompts for a LLM, similar features may apply to determining and/or generating contextual prompts for other types of models. In some embodiments, the model is configured to receive the contextual prompt from the computing node. The model may process the prompt, and the contextual information included in the prompt to create a customized message. The model may then deliver the customized message to one or more exercise devices and/or the mobile device. In some embodiments, the model may deliver the customized message to the computing node, and the computing node delivers it to the user. In some embodiments, the model is configured to generate a new activity, by modifying, adapting, or combining one or more activities stored on the exercise program library.

The one or more exercise devices and/or the mobile device may then display the customized message to the user via a display. In some embodiments, receiving the customized message may cause the exercise device to display the customized message on a display of the exercise device. In some embodiments, receiving the customized message may cause the exercise device to display the customized message on a display of the exercise device and to adjust the operating parameters of the exercise device based on the customized message. One possible benefit of delivering a customized message to a user is that the user is provided with an activity suggestion that takes into account user's injuries, goals, strengths, weaknesses, preferences, etc and provides a customized activity backed up with health data science, behavioral science, and injury recovery information.

A representation of a string diagram for creating a customized message to a user, is provided. An exercise device may transmit a request to generate a customized message to a user. In some embodiments, the request further includes user data. For example, the user data may include one or more of a training history, a goal, patterns in training, an activity level, injuries, strengths, weaknesses, motivation information, or a combination thereof.

A computing node may receive the request including the user data. The computing node may be a retrieval augmented generation (RAG) node. The computing node may, upon receiving the request, retrieve historical health data from a historical health data library. For example, the computing node may send a request for historical health data to the historical health data library. The historical health data may include one or more of exercise program information, behavioral science information, health science information, and injury recovery information. The historical health data library may send historical health data to the computing node in response to receiving the request for health data. In some embodiments, the computing node requests only health data that correlates with the user data received with the request. In some embodiments, the computing node requests all historical health data and the computing node determines the correlation between the user data and the historical health data after receiving the health data.

The computing node may then generate a contextual prompt that is based on the received user data from the exercise devices, and the received historical health data received from the historical health data library. In one or more embodiments described herein, a contextual prompt is generated based on an individual user's data and historical health data that correlates with the individual user's data. The contextual prompt may be generated through a variety of mechanisms. For example, the contextual prompt may be generated by identifying correlations by using a variety of correlation techniques to identify a correlation metric. Determining a correlation metric between the user data and historical health data may involve, for example, comparing the user's exercise data to a catalog of various different exercises and identifying similarities between them. In another example, it may involve comparing the user's prior successful motivation messages to behavioral science information related to motivation. In a further example, it may involve comparing the user's injury information to injury recovery information. In yet another example, it may involve comparing the user's goal to a nutritional information or other health science information.

The computing node may then provide the contextual prompt to a model. The model may be a generative AI model configured to generate a customized message based on an input. In some embodiments, the model is a LLM model that is trained on a large dataset. The model may include a neural network having a significant number of parameters (e.g., billions of parameters) that the model can consider in performing a task or otherwise generating an output based on the contextual prompt. The customized message created by the model may include a customized activity suggestion, a customized motivational message, or a combination thereof.

The model may then deliver the customized message to the exercise device. In some embodiments, the model delivers the customized message directly to the exercise device. In some embodiments, the model delivers the customized message to the computing node, and the computing node delivers the customized message to the exercise device. In some embodiments, receiving the customized message may cause the exercise device to display the customized message on a display of the exercise device. In some embodiments, receiving the customized message may cause the exercise device to display the customized message on a display of the exercise device and to adjust the operating parameters of the exercise device based on the customized message.

A flowchart of a series of acts or a method for creating customized messages to a user, is represented. While acts illustrate one embodiment, alternative embodiments may omit, add to, reorder, and/or modify any of the acts. The acts can be performed as part of a method. Alternatively, a computer-readable medium can comprise instructions that, when executed by one or more processors, cause a computing device to perform the acts. In some embodiments, a system can perform the acts.

The series of acts may include an act of receiving a request to generate a customized message. In some embodiments, the request may be received from a mobile device or an exercise device. In some embodiments, the request is received by a computing node. For example, the computing node may be a retrieval augmented generation (RAG) node.

The series of acts may further include an act of receiving user data. In some embodiments, the request to generate a customized message is received together with the user data. In some embodiments, the user data may include one or more of a training history, a goal, patterns in training, an activity level, injuries, strengths, weaknesses, motivation information, or a combination thereof.

The series of acts may further include an act of retrieving historical health data. In some embodiments, the historical health data may include one or more of exercise program information, behavioral science information, health science information, and injury recovery information.

The series of acts may further include an act of generating a contextual prompt wherein the contextual prompt is based on the user data and the historical health data that correlates with the user data. For example, contextual information for the contextual prompt may be identified using a variety of correlation techniques to identify a correlation metric. Determining a correlation metric between user data and historical health data may involve, for example, comparing the user's exercise data to a catalog of various different exercises and identifying similarities between them.

The series of acts may further include an act of providing the contextual prompt to a model. In some embodiments, the model is a generative AI model configured to generate an output based on the contextual prompt. In some embodiments, the model is a LLM model that is trained on a large dataset. In some embodiments, the model may include a neural network having a significant number of parameters (e.g., billions of parameters) that the model can consider in performing a task or otherwise generating an output based on the prompt.

A schematic representation of a system for training a model to create customized messages, is provided. The system may include a historical health data library. The historical health data library may further include an exercise program library. The exercise program library may include a repository of one or more exercise programs. The exercise programs may be implemented on one or more exercise devices or on a mobile device. In some embodiments, the exercise programs are specifically tailored to a particular exercise device. For example, the exercise programs may be specifically tailored to a type of exercise device, such as an elliptical. In some embodiments, the exercise programs are usable by more than one type of exercise device. For example, an exercise program may include resistance settings for a flywheel, and the exercise program may be usable by any flywheel-based exercise device. In some embodiments, the exercise programs are not tailored to be performed with an exercise machine. For example, the exercise program may be displayed on a mobile device and performed by the user without any exercise machine.

The historical health data library may further include a behavioral science library. The behavioral science library may include behavioral science information relating to motivation, habits, social influence, other types of behavioral science information, or a combination thereof. For example, the behavioral science library may include research papers, research studies, books, and any other sources of information on behavioral science. The historical health data library may further include a health science library. The health science library may include information relating to nutrition and exercise science (e.g., mechanics of movement, anatomy, exercise physiology, exercise psychology, and biomechanics). For example, the health science library may include research papers, research studies, books, and any other sources of information on nutrition and/or exercise science.

The historical health data library may further include an injury recovery library. The injury recovery library may include anatomy of injury information, rehabilitation information, kinesiology, injury treatment plans, physical therapy information, or a sports medicine information. For example, the injury recovery library may include research papers, research studies, books, and any other sources of information on injury recovery. The system may use the data stored on the historical health data library to train the model with exercise, health science, behavioral science, and injury recovery information. In one or more embodiments, the data stored on the historical health data library may be preprocessed before being delivered to the model. For example, the data may be cleaned, tokenized, split into training sets or validation sets, or a combination thereof.

The system further includes a model. In some embodiments, the model 1s a generative AI model. In some embodiments, the model is a LLM model. The model may include a neural network having a significant number of parameters (e.g., billions of parameters) that the model can consider in performing a task or otherwise generating an output based on an input. In some embodiments, the model is trained to generate a response to a prompt. The model is trained with health science, behavior science, injury recover information, exercise information, or a combination thereof. In some embodiments, the model creates new parameters consistent with the new data provided by the historical health data library.

The system further includes a validation manager. The validation manager may validate the model by utilizing cross-validation, consistency checks, and physical model evaluations to help to improve the validation and verification of the model. For example, based on the results of at least one of the cross-validation, consistency checks, and physical model evaluations, the model may be finetuned.

The system further includes a tuner. The tuner may tune one or more parameters of the model. The parameters of the model may be the parameters in the model that control the learning process. Other parameters of the model may be adjusted or derived during training of the model. For example, the tuner may identify which portions of the output received during validation of a subset of the historical health data does not correspond to the input of historical health data subset. The tuner may determine which parameter(s) may adjust the output. Adjusting or tuning the parameters may adjust the output. The tuner may adjust the parameters of the model until the validation manager successfully validates all subsets of the historical health data.

In some embodiments, the trained model is configured to generate a new activity, by modifying, adapting, or combining one or more activities used for training the model. In some embodiments, a trained model may be used without a RAG. For example, a trained model that includes the knowledge of a user data and historical health data may not require a RAG to provide any additional contextual information or a prompt for the trained model to create a customized message. In some embodiments, a trained model may be used together with a RAG. For example, the trained model may be trained with the historical health data, while the RAG may still provide contextual prompt to the trained model that includes the user data.

A flowchart of a series of acts or a method for creating customized messages to a user, is provided. While the acts illustrate one embodiment, alternative embodiments may omit, add to, reorder, and/or modify any of the acts. The acts can be performed as part of a method. Alternatively, a computer-readable medium can comprise instructions that, when executed by one or more processors, cause a computing device to perform the acts. In some embodiments, a system can perform the acts.

The series of acts may include an act of receiving a plurality of historical health data. In some embodiments, the plurality of historical health data is divided into a plurality of subsets.

The series of acts may further include an act of providing the historical health data as input for a model. In some embodiments, the historical health data may be preprocessed before provided as input for the model. The model may include plurality of parameters.

The series of acts may further include an act of performing validation for the model. In some embodiments, performing validation for the model includes one or more of a cross-validation, a consistency check, or a physical model evaluation.

The series of acts may further include an act of finetuning the model. For example, finetuning the model may include adjusting one or more of the plurality of parameters.

The series of acts may further include an act of validating the model. In some embodiments, the model is validated when all subsets of the plurality of historical health data match with an output received from the model.

Following are sections in accordance with at least one embodiment of the present disclosure:
A1. A method for creating a customized message to a user, comprising:
   receiving a request to generate the customized message to the user;
   receiving user data;
   retrieving historical health data that correlates with the user data;
   generating a contextual prompt for a model, wherein the contextual prompt is based on the user data and the historical health data that correlates with the user data; and
   providing the contextual prompt to the model, wherein the contextual prompt causes the model to create the customized message.
A2. The method of section A1, wherein the customized message is a customized activity suggestion.
A3. The method of section A2, wherein the customized activity suggestion includes one or more of an exercise, an exercise length, an intensity level of the exercise, an exercise date, an exercise start time, recommended recovery time, or dietary information.
A4. The method of sections A1 and A2, wherein the customized message is a customized motivational message.
A5. The method of any of sections A1-A4, wherein the request to generate the customized message is received from a mobile device.
A6. The method of any of sections A1-AS, wherein the user data is received from an exercise device.
A7. The method of any of sections A1-A6, wherein the user data includes at least one or more of a training history, a goal, patterns in training, an activity level, injuries, strengths, weaknesses, or motivation information.
A8. The method of section A7, wherein the patterns in training include one or more of, a time spent on an activity, a time between activities, preferred weekdays for performing an activity, or preferred hours for performing an activity.
A9. The method of any of sections A7-A8, wherein the training history includes all activities performed by the user.
A10. The method of any of section A7-A9, wherein the strengths and/or weaknesses are identified by the user.
A11. The method of any of sections A7-A11, wherein the strengths and/or weaknesses are automatically identified by an exercise device or a mobile device.
A12. The method of any of sections A1-A11, wherein the historical health data includes one or more of exercise programs, behavioral science information, behavioral health science, or injury recovery information.
A13. The method of any of sections A1-A12, wherein generating the contextual prompt includes generating the contextual prompt with a retrieval augmented generation (RAG) system.
A14. The method of any of section A1-A13, wherein the model is a large language model (LLM).
A15. The method of any of section A1-A14, further comprising delivering the customized message to the user.
B1. A computing device for facilitating creating a customized message to a user, comprising:
   a processor and memory, the memory including instructions that cause the processor to:
   receive a request to generate the customized message to the user;
   receive user data;
   retrieve historical health data that correlate with the user data;
   generate a contextual prompt for a model wherein the contextual prompt is based on the user data and the historical health data that correlates with the user data; and
   provide the contextual prompt to the model, wherein the contextual prompt causes the model to create the customized message.
B2. The computing device of section B1, wherein the customized message 1s a customized activity suggestion.
B3. The computing device of any of sections B1-B2, wherein the historical health data includes one or more of exercise programs, behavioral science information, behavioral health science, or injury recovery information.
C1. A method for training a model, comprising:
   receiving a plurality of historical health data, wherein the plurality of historical health data is divided into plurality of subsets;
   providing the plurality of subsets as input for the model, wherein the model includes plurality of parameters;
   performing a validation for the model, wherein performing a validation for the model includes one or more of a cross-validation, a consistency check, or a physical model evaluation;
   finetuning the model by adjusting one or more of the plurality of parameters; and
   validating the model when all subsets of the plurality of historical health data match with an output received from the model.
C2. The method of section C1, wherein receiving a plurality of historical health data further includes preprocessing the plurality of historical health data.

Certain components that may be included within a computer system, such as the computing node as previously discussed, is presented. One or more computer systems may be used to implement the various devices, components, and systems described herein.

The computer system includes a processor. The processor may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor may be referred to as a central processing unit (CPU). The computer system may have a single processor or in an alternative configuration, a combination of processors (e.g., an ARM and DSP) could be used.

The computer system also includes memory in electronic communication with the processor. The memory may be any electronic component capable of storing electronic information. For example, the memory may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions and data may be stored in the memory. The instructions may be executable by the processor to implement some or all of the functionality disclosed herein. Executing the instructions may involve the use of the data that is stored in the memory. Any of the various examples of modules and components described herein may be implemented, partially or wholly, as instructions stored in memory and executed by the processor. Any of the various examples of data described herein may be among the data that is stored in memory and used during execution of the instructions by the processor.

A computer system may also include one or more communication interfaces for communicating with other electronic devices. The communication interface(s) may be based on wired communication technology, wireless communication technology, or both. Some examples of communication interfaces include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system may also include one or more input devices and one or more output devices. Some examples of input devices include a keyboard, mouse, microphone, remote control device, button, joystick, trackball, touchpad, and lightpen. Some examples of output devices include a speaker and a printer. One specific type of output device that is typically included in a computer system is a display device. Display devices used with embodiments disclosed herein may utilize any suitable image projection technology, such as liquid crystal display (LCD), light-emitting diode (LED), gas plasma, electroluminescence, or the like. A display controller may also be provided, for converting data stored in the memory into text, graphics, and/or moving images (as appropriate) shown on the display device.

The various components of the computer system may be coupled together by one or more buses, which may include a power bus, a control signal bus, a status signal bus, a data bus, etc.

Embodiments of the present disclosure may thus utilize a special purpose or general-purpose computing system including computer hardware, such as, for example, one or more processors and system memory. Embodiments within the scope of the present disclosure also include physical and other computer-readable media for carrying or storing computer-executable instructions and/or data structures, including applications, tables, data, libraries, or other modules used to execute particular functions or direct selection or execution of other modules. Such computer-readable media can be any available media that can be accessed by a general purpose or special purpose computer system. Computer-readable media that store computer-executable instructions (or software instructions) are physical storage media. Computer-readable media that carry computer-executable instructions are transmission media. Thus, by way of example, and not limitation, embodiments of the present disclosure can include at least two distinctly different kinds of computer-readable media, namely physical storage media or transmission media. Combinations of physical storage media and transmission media should also be included within the scope of computer-readable media.

Both physical storage media and transmission media may be used temporarily store or carry, software instructions in the form of computer readable program code that allows performance of embodiments of the present disclosure. Physical storage media may further be used to persistently or permanently store such software instructions. Examples of physical storage media include physical memory (e.g., RAM, ROM, EPROM, EEPROM, etc.), optical disk storage (e.g., CD, DVD, HDDVD, Blu-ray, etc.), storage devices (e.g., magnetic disk storage, tape storage, diskette, etc.), flash or other solid-state storage or memory, or any other non-transmission medium which can be used to store program code in the form of computer-executable instructions or data structures and which can be accessed by a general purpose or special purpose computer, whether such program code is stored as or in software, hardware, firmware, or combinations thereof.

A “network” or “communications network” may generally be defined as one or more data links that enable the transport of electronic data between computer systems and/or modules, engines, and/or other electronic devices. When information is transferred or provided over a communication network or another communications connection (either hardwired, wireless, or a combination of hardwired or wireless) to a computing device, the computing device properly views the connection as a transmission medium. Transmission media can include a communication network and/or data links, carrier waves, wireless signals, and the like, which can be used to carry desired program or template code means or instructions in the form of computer-executable instructions or data structures and which can be accessed by a general purpose or special purpose computer.

Further, upon reaching various computer system components, program code in the form of computer-executable instructions or data structures can be transferred automatically or manually from transmission media to physical storage media (or vice versa). For example, computer-executable instructions or data structures received over a network or data link can be buffered in memory (e.g., RAM) within a network interface module (NIC), and then eventually transferred to computer system RAM and/or to less volatile physical storage media at a computer system. Thus, it should be understood that physical storage media can be included in computer system components that also (or even primarily) utilize transmission media.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for creating a customized message to a user, comprising:
receiving a request to generate the customized message to the user;
receiving user data;
retrieving historical health data that correlates with the user data;
generating a contextual prompt for a model, wherein the contextual prompt is based on the user data and the historical health data that correlates with the user data; and
providing the contextual prompt to the model, wherein the contextual prompt causes the model to create the customized message.

2. The method of claim 1, wherein the customized message comprises a customized activity suggestion.

3. The method of claim 2, wherein the customized activity suggestion comprises an exercise, an exercise length, an intensity level of the exercise, an exercise date, an exercise start time, recommended recovery time, dietary information, or any combination thereof.

4. The method of claim 1, wherein the customized message comprises a customized motivational message.

5. The method of claim 1, wherein receiving the request to generate the customized message comprises:
receiving the request from a mobile device.

6. The method of claim 1, wherein receiving the user data comprises:
receiving the user data from an exercise device.

7. The method of claim 1, wherein the user data comprises a training history, a goal, patterns in training, an activity level, injuries, strengths, weaknesses, or motivation information.

8. The method of claim 7, wherein the patterns in training comprise a time spent on an activity, a time between activities, preferred weekdays for performing an activity, preferred hours for performing an activity, or any combination thereof.

9. The method of claim 7, wherein the training history comprises one or more activities performed by the user.

10. The method of claim 7, wherein the strengths, the weaknesses, or both are identified by the user.

11. The method of claim 7, wherein the strengths, the weaknesses, or both are automatically identified by an exercise device or a mobile device.

12. The method of claim 1, wherein the historical health data comprises exercise programs, behavioral science information, behavioral health science, injury recovery information, or any combination thereof.

13. The method of claim 1, wherein generating the contextual prompt comprises:
generating the contextual prompt with a retrieval augmented generation (RAG) system.

14. The method of claim 1, wherein the model is a large language model (LLM).

15. The method of claim 1, further comprising:
delivering the customized message to the user.

16. A computing device for facilitating creating a customized message to a user, comprising:
one or more processors and one or more memories, the one or more memories comprising instructions which, when executed by the one or more processors, cause the computing device to:
receive a request to generate the customized message to the user;
receive user data;
retrieve, a historical health data that correlate with the user data;
generate a contextual prompt for a model wherein the contextual prompt is based on the user data and the historical health data that correlates with the user data; and
provide the contextual prompt to the model, wherein the contextual prompt causes the model to create the customized message.

17. The computing device of claim 16, wherein the customized message comprises a customized activity suggestion.

18. The computing device of claim 16, wherein the historical health data comprises exercise programs, behavioral science information, behavioral health science, injury recovery information, or any combination thereof.

19. A method for training a model, comprising:
receiving a plurality of historical health data, wherein the plurality of historical health data is divided into plurality of subsets;
providing the plurality of subsets as input for the model, wherein the model includes plurality of parameters;
performing a validation for the model, wherein performing the validation for the model comprises a cross-validation, a consistency check, a physical model evaluation, or any combination thereof;
adjusting one or more of the plurality of parameters to fine-tune the model; and
validating the model when all subsets of the plurality of historical health data match with an output received from the model.

20. The method of claim 19, wherein receiving the plurality of historical health data comprises:
preprocessing the plurality of historical health data.
